(19) United States
(12) Patent Application Publication
(10) Pub. No.: US 2015/0313837 A1
(43) Pub. Date: Nov. 5, 2015

(54) POLYMERIC HYDROGEL COMPOSITIONS WHICH RELEASE ACTIVE AGENTS IN RESPONSE TO ELECTRICAL STIMULUS

(71) Applicant: University of Witwatersrand, Johannesburg, Johannesburg (ZA)

(72) Inventors: Thomas Tsai, Johannesburg (ZA); Viness Pillay, Johannesburg (ZA); Yahya Essop Choonara, Johannesburg (ZA); Lisa Claire Du Toit, Johannesburg (ZA)

(21) Appl. No.: 14/678,618

(22) Filed: Apr. 3, 2015

Related U.S. Application Data
(63) Continuation-in-part of application No. 13/989,435, filed on Sep. 5, 2013, now abandoned.

(30) Foreign Application Priority Data
Nov. 26, 2010 (ZA) 2010/03746

(51) Int. Cl. A61K 9/00; A61K 41/00; A61K 47/32; A61K 31/405; A61K 47/34

(52) U.S. Cl. CPC A61K 9/0024; A61K 31/405; A61K 47/34; A61K 47/32; A61K 41/00

(57) ABSTRACT

A polymeric hydrogel composition is described for the delivery of a pharmaceutically active agent when an electrical stimulus is applied to the composition. The composition comprises a polymer which forms the hydrogel, such as poly vinyl alcohol (PVA) cross-linked with diethyl acetamidomalonate (DAA), an electroactive polymer such as polyaniline and a pharmaceutically active agent such as an analgesic, and in particular, indomethacin. The composition can be subcutaneously implanted at a targeted site and under normal conditions, the active agent will be entrapped in the hydrogel itself. However, upon the application of an electric current to the hydrogel, the active agent will be released. When the electric current is removed, the change is reversed and the active agent will cease to be released. In one embodiment of the invention, the hydrogel composition is for use in alleviating chronic pain.

### FIELD OF THE INVENTION

The invention relates to a polymeric hydrogel composition containing a pharmaceutically active agent or drug which can be implanted subcutaneously at a target site and which is capable of drug release via stimulus activation from an external device.

### BACKGROUND TO THE INVENTION

The management of chronic pain has always proved to be challenging, both for clinicians and patients. The pain arises due to the activation of nociceptors, which convey signals to the brain and are then interpreted as pain (Semenchuk, 2000). This activation may be caused by injury or dysfunction of the neurons. In most cases, relieving the pain completely is rare and difficult. The World Health Organisation (WHO) has set up a three-step ladder algorithm as a guide for the treatment of pain. The ladder aims to treat pain by using a combination of non-opioid analgesics and opioid analgesics and proves to be effective for 80-90% of the cases. However, treatment with such analgesics and opioids results in significant side-effects. Patients may feel severe chronic nausea, vomiting, itching, constipation or drowsiness. In severe cases, patient dependence and addiction may occur, leading to treatment complications. Conventional treatment of chronic pain includes patientcontrolled pump administration of oral tablets and drugs which relies on patient compliance and often induces gastric side-effects. Long-term use of non-steroidal anti-inflammatory drugs (NSAIDs) may cause gastric ulceration, increased cardiovascular risk, fluid retention and interactions with anti-coagulants. Oral drugs may have limited dissolution or be strongly ionized which decreases absorption through the intestine. Traditional oral or parenteral drugs may not have adequate therapeutic effects and further metabolism and inactivation of the drug may lower the systemic levels of drug even further.

### SUMMARY OF THE INVENTION

According to a first embodiment of the invention, there is provided a polymeric hydrogel composition for the delivery of a pharmaceutically active agent to a human or animal when an electrical stimulus is applied to the composition, the composition comprising:
a polymer which forms a hydrogel;
an electroactive polymer; and
a pharmaceutically active agent;

wherein the pharmaceutically active agent is released from the hydrogel composition when the electric current is applied to the hydrogel composition.

The electrical stimulus may be an electric current.

The polymer which forms the hydrogel may be poly vinyl alcohol (PVA), and may be cross-linked with a cross-linking agent. The cross-linking agent may be diethyl acetamidomalonate (DAA). The cross-linking agent may be Eudragit. The Eudragit may be at least one of the following group: Eudragit RL 100, Eudragit L 100-55, Eudragit S100, Eudragit E100, and Eudragit RS.

The electroactive polymer may be polyaniline, polypyrrole or polythiophene, and is preferably polyaniline. Preferably, the electroactive polymer may be emeraldine polyaniline.

The hydrogel composition may further comprise polyethylene glycol (PEG).

The hydrogel composition may further comprise a polymerization initiator. The polymerization initiator may be ammonium persulfate.

The hydrogel composition may be for use in relieving or ameliorating chronic pain, and the pharmaceutically active agent may be an analgesic, and is preferably a non-steroidal anti-inflammatory drug (NSAID) such as indomethacin.

The pharmaceutically active agent may cease to be released when the current is no longer applied to the hydrogel composition.

The hydrogel composition may be in an implantable form and is preferably biodegradable.

The hydrogel composition may provide controlled and targeted delivery of the pharmaceutically active agent. The current may be applied for a time period of from less than about 1 second to about 60 seconds, more preferably from about 1 second to about 5 seconds or from about 30 seconds to about 60 seconds.

The potential difference which is applied may be from about 0.3 volts to about 0.5 volts.

According to a second embodiment of the invention, there is provided a method of preparing a hydrogel composition which is capable of delivering a pharmaceutically active agent to a human or animal when an electrical stimulus is applied to the hydrogel composition, the method comprising the steps of:
mixing a polymer for forming a hydrogel, a cross-linking agent, an electroactive polymer and a pharmaceutically active agent; and
allowing a hydrogel composition to form which contains the electroactive agent and pharmaceutically active agent.

According to a third embodiment of the invention, there is provided a method of treating chronic pain in a human or animal, the method comprising the steps of:
implanting a hydrogel composition substantially as described above in the human or animal at a targeted site of delivery; and
applying an electrical stimulus to the hydrogel composition to release a dose of a pharmaceutically active agent from the hydrogel composition.

### BRIEF DESCRIPTION OF THE FIGURES

FIG. 1: shows the apparatus that was used to determine drug release (indomethacin) of a hydrogel composition of the present invention under electric current.

FIG. 2: shows a fractional drug release profile of a hydrogel composition over a three hour period with 45 seconds of electric current at an hourly interval.

FIG. 3: shows the amount of drug released by a hydrogel composition when exposed to various potential differences.

FIG. 4: shows a hydrogel composition before exposure to an electric current.

FIG. 5: shows a hydrogel composition after exposure to an electric current.

FIG. 6: shows the erosion of a hydrogel composition after 60 sampling time-points.

FIG. 7: shows an FTIR graph of a hydrogel composition without indomethacin.

FIG. 8: shows an FTIR profile of an eroded hydrogel composition containing indomethacin.

FIG. 9: shows a light microscopy image of a first erosion site of a hydrogel compostion under 32× magnification.

FIG. 10: shows a light microscopy image of a second erosion site of a hydrogel compostion under 32× magnification.

FIG. 11: shows the surface morphology of an uneroded hydrogel system using a scanning electron microscope (SEM).

FIG. 12: shows the surface morphology of an eroded hydrogel system using a SEM.

FIG. 13: shows a typical intensity profile obtained by a ZetaSizer indicating the presence and the size distribution of nano-spheres within the hydrogel composition.

FIG. 14: shows a hydrogel composition with 0.25 g poly vinyl alcohol (PVA) under 32× magnification.

FIG. 15: shows a hydrogel composition with 0.5 g PVA under 32× magnification.

FIG. 16: shows a hydrogel composition with 1 g PVA under 32× magnification.

FIG. 17: shows the force required to compress a hydrogel composition with no diethyl acetamidomalonate (DAA).

FIG. 18: shows the force required to compress a hydrogel composition with 0.25 g DAA.

FIG. 19: shows the force required to compress a hydrogel composition with 1 g DAA.

FIG. 20: shows the drug release from a hydrogel composition without DAA.

FIG. 21: shows the drug release from a hydrogel composition with DAA.

FIG. 22: shows the proposed mechanism of drug release from the hydrogel composition.

FIG. 23: is a schematic showing the design of the in vivo studies.

### DETAILED DESCRIPTION OF THE INVENTION

A polymeric hydrogel composition is described for the delivery of a pharmaceutically active agent or drug to a human or animal when an electrical current is applied to the hydrogel composition. The hydrogel composition comprises a polymer which forms a hydrogel, an electroactive polymer and a pharmaceutically active agent or drug. The hydrogel composition is typically biodegradable and can be subcutaneously implanted into the human or animal at a targeted site and under normal conditions, the active agent will be entrapped in, attached to or adsorbed onto the hydrogel itself. However, upon the application of a stimulus to the hydrogel, such as an electric current, the hydrogel will undergo structural changes and the active agent will be released into the blood stream of the human or animal. When the electric current is removed, the change is reversed and thus the active agent will cease to be released from the hydrogel composition. The hydrogel composition of the invention will in some instances be referred to as a drug-entrapped electro-liberated polymeric hydrogel system (EPHS). The hydrogel composition of the invention will in some instances be referred to as a stimuli-actuated polymeric device (SAPD).

In one embodiment of the invention, the hydrogel composition is for use in the controlled and targeted delivery of a pharmaceutically active agent into the surrounding tissue for the alleviation of chronic pain. The pharmaceutically active agent is typically an analgesic such as acetaminophen or a non-steroidal anti-inflammatory drug (NSAID). NSAIDs include Aspirin (Anacin, Ascriptin, Bayer, Bufferin, Ecotrin, Excedrin), choline and magnesium salicylates (CMT, Tricosal, Trilisate), Choline salicylate (Arthropan), Celecoxib (Celebrex), Diclofenac potassium (Cataflam), Diclofenac sodium (Voltaren, Voltaren XR), Diclofenac sodium with misoprostol (Arthrotec), Diflunisal (Dolobid), Etodolac (Lodine, Lodine XL), Fenoprofen calcium (Nalfon), Flurbiprofen (Ansaid), Ibuprofen (Advil, Motrin, Motrin IB, Nuprin), Indomethacin (Indocin, Indocin SR), Ketoprofen (Actron, Orudis, Orudis KT, Oruvail), Magnesium salicylate (Arthritab, Bayer Select, Doan's Pills, Magan, Mobidin, Mobogesic), Meclofenamate sodium (Meclomen), Mefenamic acid (Ponstel), Meloxicam (Mobic), Nabumetone (Relafen), Naproxen (Naprosyn, Naprelan), Naproxen sodium (Aleve, Anaprox), Oxaprozin (Daypro), Piroxicam (Feldene), Rofecoxib (Vioxx), Salsalate (Amigesic, Anaflex 750, Disalcid, Marthritic, Mono-Gesic, Salflex, Salsitab), Sodium salicylate (various generics), Sulindac (Clinoril), Tolmetin sodium (Tolectin) and Valdecoxib (Bextra). A particularly suitable NSAID is indomethacin. The hydrogel composition can include more than one pharmaceutically active agent or drug. The pharmaceutically active agent or drug can be loaded onto or into micro- or nano-particles.

The hydrogel can be formed from poly vinyl alcohol (PVA) cross-linked with diethyl acetamidomalonate (DAA). This cross-linking can result in a hydrogel with an irregular shape. Eudragit may also be used as a cross-linking agent.

The electroactive polymer is an electrical stimulus actuated polymer such as polyaniline (PANi), polypyrrole or polythiophene, and is typically polyaniline. The polyaniline may be in at least one of the following oxidation states: pernigraniline, emeraldine and leucoemeraldine. The Applicant has surprisingly found that emeraldine base polyaniline provides the highest stability and that upon doping, the emeraldine salt form is preferred as it provides the favorable electrical conduction properties especially when formulated with the polyvinyl alcohol hydrogel. Electrical stimulus actuated polymers are polymers which undergo structural or behaviour changes when exposed to an electric current or potential difference. Electroactive polymers (EAP) have previously been used as biosensors and in the field of robotics. EAPs such as polyaniline, polypyrrole and poly thiophene are well-researched conducting polymers due to their easy synthesis and rich redox reaction. Their drawback, however, is their poor mechanical property.

The hydrogel composition may comprise from about 0.5 g to about 0.8 g PVA, from about 0 g to about 0.30 g DAA and from about 1.0% w/w to about 4% w/w PANi. Particularly the PANi (polyaniline is the emeraldine base polyaniline).

The hydrogel composition may further comprise polyethylene glycol (PEG).

The hydrogel composition may further comprise a polymerization initiator. The polymerization initiator may be ammonium persulfate.

The potential difference which is applied may be from about 0.3 volts to about 0.5 volts.

The EAP-based drug delivery system of the present invention can be implanted subcutaneously at a target site and can be capable of drug release via stimulus activation from an external device. For example, a small electrical supply device with, for example, a 1.5 volt battery, could be worn by the user over or in the region of where the composition has been implanted. The user could activate the electrical supply device at the push of a button to send a current through the skin to the composition. The electrical supply device could include a means, e.g. an electronic chip, to control the number of doses that a patient can take a day.

The invention will now be described in more detail by way of the following non-limiting examples.

### EXAMPLES

### Example 1

Materials

Poly vinyl alcohol was used to form a hydrogel. Diethyl acetamidomalonate (DAA) was used as a crosslinker for increasing the structural integrity of the hydrogel. A conducting polymer, polyaniline (PANi), was used to ensure that electric current is conducted throughout the entire hydrogel and thus ensures a more rapid, consistent response from the hydrogel. However, other electroative polymers (EAPs), such as polypyrrole or polythiophene, could also be used. Indomethacin was used as a model drug. The PANi used was the PANi emeraldine base, M_{w} 20 000. The PVA (M_{w} 88 000) and the indomethacin were purchased from Sigma Chemical Company (St Louis, Mo., USA). The DAA had a purity of >98% and was purchased from Fluka Chemie AG (Buchs, Switzerland).

Preparation of the Hydrogel Composition

The poly vinyl alcohol (PVA) and diethyl acetamidomalonate (DAA) were mixed together in a 1:1 w/w ratio. The poly vinyl alcohol, M_{w} approx 88 000, (0.5 g) was dissolved in 10 mL boiling water and allowed to cool for fifteen minutes. DAA (0.5 g), 2% w/w PANi and indomethacin (100 mg) were dissolved in 10 mL acetone until fully dissolved. The dissolved DAA solution was then added into the cooled PVA solution and stirred with a glass rod for one minute until all the polymers had reacted and a drug-loaded hydrogel had formed on the tip of the glass rod. Several other hydrogels with different ratios of PVA: DAA and different molecular weights of PVA were also prepared.

Assessment of Drug Release from the Polymeric Hydrogel in the Presence of an Electric Current

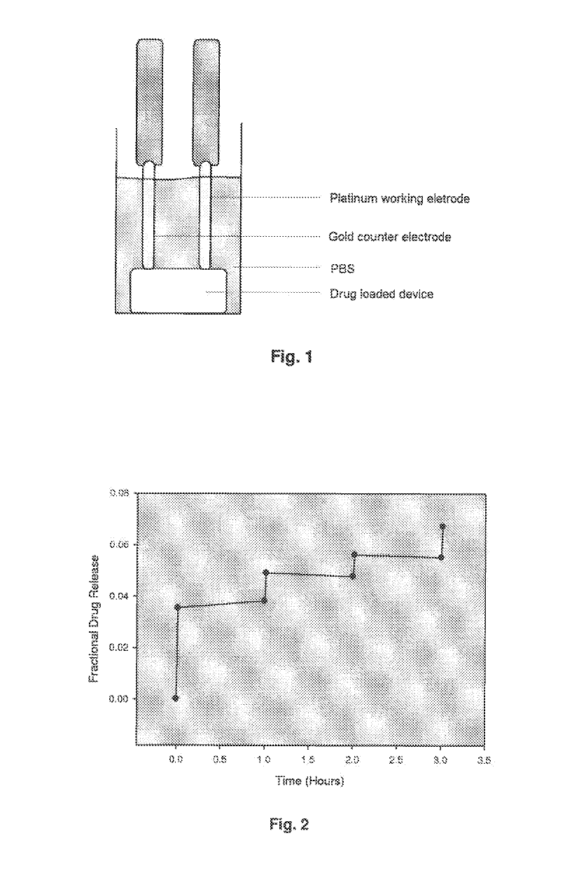

The drug-loaded hydrogels were subjected to an electric current in phosphate buffered saline (PBS) in order to assess release of the drug. This was done by placing the hydrogels into 40 mL of PBS and allowing a potential difference of 1.2 V with a current of 0.3 A to pass through the PBS. The equipment used was a PGSTAT 302N potentiostat/glavanostat (Autolab, Utrecht, Netherlands) with platinum as the working electrode and gold as the counter electrode. The setup of the experiment is depicted in FIG. 1.

An electric current was passed through the hydrogel for 45 seconds and 1 mL samples were then taken. This was repeated three times, after which the samples were scanned via UV/visible spectroscopy for any presence of the drug.

Assessment of Indomethacin Release from the Hydrogels in the Presence and the Absence of an Electric Current

The indomethacin-loaded hydrogels were left in 40 mL PBS for 12 hours, and a 1 mL sample was then taken in order to assess for any drug release prior to exposure to an electric current. The results obtained from the UV/visible spectroscopy indicated that there was no drug present in the sample. Further tests for drug release of the indomethacin-loaded hydrogels in the presence of an electric current were performed. The results are summarized in Table 1.

TABLE 1
Indomethacin release from the PANi-hydrogel
system when exposed to electric current

| | 45 | 90 | 135 | 180 | 225 |
|---|---|---|---|---|---|
| | seconds | seconds | seconds | seconds | seconds |
| UV absorbance | 0.0204 | 0.0500 | 0.0114 | 0.0134 | 0.0158 |
| Drugs in mg | 0.1200 | 0.1778 | 0.1022 | 0.1061 | 0.1108 |

These results show that drug release was achieved when the hydrogels were placed under an electric current. The hydrogels were also assessed in order to ensure that drug leakage did not occur once the hydrogels had been exposed to the electric current due to any possible structural changes which may have occurred. The system was therefore left in 50 mL of PBS for 12 hours, and 1 mL sample was taken and assessed for any presence of drugs. The results obtained from the UV Ivisible spectroscopy indicated that there was no drug leakage. This suggests that an indomethacin-loaded hydrogel could be used for the purpose of an electroactive drug delivery system. The hydrogels were then then assessed for their drug release capacity. They were once again immersed in PBS and an electric current was passed through them. This time, 35 samples were extracted and assessed by UV/visible spectroscopy for the amount of drugs which were released. The results are shown in Table 2.

TABLE 2
Amount of indomethacin released by hydrogels (35 samples)

| Sample | Drugs (mg) |
|---|---|
| 1 | 0.081 |
| 2 | 0.085 |
| 3 | 0.096 |
| 4 | 0.098 |
| 5 | 0.118 |
| 6 | 0.084 |
| 7 | 0.084 |
| 8 | 0.084 |
| 9 | 0.082 |
| 10 | 0.082 |
| 11 | 0.135 |
| 12 | 0.139 |
| 13 | 0.148 |
| 14 | 0.160 |
| 15 | 0.158 |
| 16 | 0.083 |
| 17 | 0.082 |
| 18 | 0.082 |
| 19 | 0.082 |
| 20 | 0.083 |
| 21 | 0.121 |
| 22 | 0.103 |
| 23 | 0.101 |
| 24 | 0.103 |
| 25 | 0.097 |
| 26 | 0.109 |
| 27 | 0.107 |
| 28 | 0.109 |
| 29 | 0.099 |
| 30 | 0.107 |
| 31 | 0.103 |
| 32 | 0.097 |
| 33 | 0.103 |
| 34 | 0.103 |
| 35 | 0.097 |

The amount of drug released ranged from 0.081 mg to 0.160 mg. The hydrogel was then assessed one last time for any leakage of drugs. The hydrogel was immersed in 50 mL of PBS for 12 hours. A 1 mL sample was taken and the UV absorbance indicated that there was no leakage of indomethacin when the hydrogel was left immersed in the absence of electricity.

One challenge with an electroactive hydrogel device such as this is that its response may slowly lag in time. As can be seen in Table 2, there is a slight difference in drug release from the first ten samples as compared to the last ten samples. This is probably due to the slightly lagged response from the hydrogel when it was left immersed and unused in PBS. This phenomenon is possibly due to the ion exchange between the hydrogel and the surrounding medium, which tends to diminish the electrochemical control of the drug release (Lira, 2005). The last step in this study was to determine how much drug could be released before the hydrogel became totally depleted of drug. The hydrogel was therefore continuously exposed to an electric current and samples were assessed for drug until no more drugs were released. The results are indicated in Table 3.

TABLE 3
The drug released from the PANi-hydrogel system. From sample
68 onwards, the drug released dropped to a negligible value

| Sample | Drugs (mg) |
|---|---|
| 36 | 0.100 |
| 37 | 0.105 |
| 38 | 0.101 |
| 39 | 0.086 |
| 40 | 0.107 |
| 41 | 0.121 |
| 42 | 0.099 |
| 43 | 0.139 |
| 44 | 0.119 |
| 45 | 0.113 |
| 46 | 0.148 |
| 47 | 0.168 |
| 48 | 0.141 |
| 49 | 0.121 |
| 50 | 0.143 |
| 51 | 0.088 |
| 52 | 0.088 |
| 53 | 0.088 |
| 54 | 0.090 |
| 55 | 0.141 |
| 56 | 0.096 |
| 57 | 0.090 |
| 58 | 0.090 |
| 59 | 0.088 |
| 60 | 0.086 |
| 61 | 0.097 |
| 62 | 0.088 |
| 63 | 0.088 |
| 64 | 0.096 |
| 65 | 0.099 |
| 66 | 0.088 |
| 67 | 0.097 |
| 68 | 0.000 |
| 69 | 0.000 |
| 70 | 0.000 |

Diclofenac sodium, ibuprofen and indomethacin were used and results indicated that indomethacin was the only suitable drug for this implantable hydrogel, as no leakage occurred when an electric current was not applied to hydrogels containing indomethacin. One possible explanation for this phenomenon is the larger molecular size of indomethacin as compared to diclofenac sodium and ibuprofen. This larger molecular size means that indomethacin is better entrapped inside the three dimensional network of the hydrogel system. Although most diclofenac sodium and ibuprofen molecules were well entrapped in the centre of the hydrogel, the drug leakage may still have occurred on the surface. Since the drug is entrapped in the hydrogel system, it is possible to suggest a release mechanism of passive diffusion outwards of the hydrogel.

Optimization of the Hydrogels

Following on from the design of the hydrogels, the next step was to determine the various factors which affected the hydrogels, thus allowing optimization thereof. These factors included internal factors such as the ratio of the constituent polymers, and external factors such as the environmental pH and temperature, as they could affect the physico-chemical or physico-mechanical properties of the hydrogels.

In order to determine the optimum working range of the hydrogels, the internal factors such as a variation in the ratio of constituents and the amount of drugs used were first assesssed. By varying the ratio of constituents, the rate of release of the drugs and the physicomechanical properties of the hydrogel can be altered. The crosslinking should be sufficiently adequate to provide good structural integrity while not hindering drug release significantly. The amount of drugs loaded into the hydrogel should be maximized so that more drug release may be achieved, thereby prolonging the lifespan of the hydrogel. Preliminary results had indicated that the higher the erosion rate, the higher the amount of drug that should be present. Therefore, a good starting point for the testing of this hydrogel system was to begin with a hydrogel with high PANi concentration, high drug loading and intermediate volume. This should yield a high erosion rate while still maintaining the structural integrity of the system. In order to ensure that the hydrogel system that was synthesized was desirable, computer simulation was also performed to ensure that the optimum ratio was chosen. Once the internal factors were established, the hydrogel system was further characterized for its drug release rate under different environmental factors.

All of the tests were initially carried out under physiological pH of 7.4. However, when an infection occurs in the human body, the surrounding tissue becomes acidic. This is a result of anaerobic glycolysis by the bacteria, resulting in lactic acid at the infection site (McCormick, 1983). Furthermore, the blood stasis caused by the infection causes a build-up of carbon dioxide which decreases the pH level even further (Menkin, 1956). It was therefore important to determine whether this change in environmental pH can affect the drug release rate of the hydrogel. Other environmental factors such as temperature and current strength were also investigated in order to determine what affect these factors have on drug release. For example, a change in temperature may affect the visco-elastic property of the hydrogel. This change in physico-mechanical property may, in turn, affect the erosion rate and thus the rate of drug release. Other characterizations included properties such as melting points, glass transition temperature and thermal degradation.

Optimization of the Potential Difference to be Applied to the Hydrogel System in Order to Achieve an Ideal Drug Release Profile

Taking into account the effects that various polymers have on the hydrogel system, a hydrogel system with minimal crosslinking, intermediate volume and high PANi concentration was a favourable starting point for the synthesis of the hydrogel system. A hydrogel composition was therefore synthesized using 0.5 g PVA, 0.5 g 2% w/w PANi and 100 mg indomethacin. The DEE for this hydrogel was 70.25%. The testing conditions were first standardized. Thus far, all the experiments had been carried out at room temperature under 1.2V for 45 seconds. Therefore, an experiment was conducted by immersing the hydrogel system in 20 mL of PBS followed by exposure to an electric current for 45 seconds. The hydrogel was then left in the PBS for an hour before another electric current was passed through the PBS. Samples were taken before and after the electric current in order to assess the amount of drug released and if there was any leakage of drugs during the absence of the electric current. This experiment was conducted over three hours in order to assess the response of the hydrogel system under these circumstances.

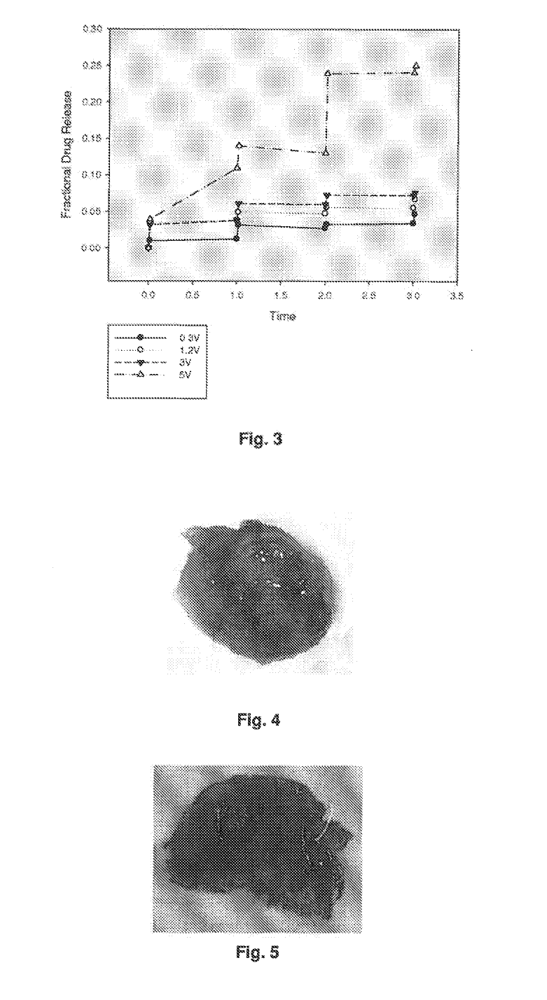

From FIG. 2, it is evident that the hydrogel is capable of a burst release of drug in the presence of an electric current, although the initial release was higher than the rest. The stepwise increase in drug is an indication of a favourable drug release profile because it demonstrates significantly increased drug release when the hydrogel system was exposed to electric current for the short amount of time. However, the amount of drug release should ideally be higher than what was seen. The effects that a potential difference has on the PANi-hydrogel system were therefore determined. Hydrogels were synthesized and exposed to a potential difference of 0.3V, 3V and 5V. The drug release profiles were then assessed and compared to the drug release profile of the hydrogel system under 1.2V so as to determine any difference in terms of drug release behaviour and response caused by a difference in the voltage applied. The results are summarized in FIG. 3, which shows fractional drug release against time when the PANi-hydrogel system is exposed to various potential differences.

From the results shown in FIG. 3, it can be seen that the higher the potential difference applied, the more the drug was released. Thus, by choosing the optimal potential difference, it is possible to achieve a release of a therapeutic dose of indomethacin while controlling the amount of drugs to be released at every interval so as not to have an excessive amount of drug released. A high fractional drug release from the PANi-hydrogel system during a short amount of time means that the implant will have to be replaced frequently and is thus unfavourable.

The Drug Release Mechanism of the PANi-Hydrogel

Murdan (2003) has suggested methods by which drugs are released via electro-responsive methods. These methods are forced eviction of drug due to deswelling; electrophoresis of drugs towards charged electrodes; and erosion of hydrogel leading to liberation of drugs. The drug release mechanism from the hydrogel of the present invention may be one of these three possible mechanisms. When the hydrogel system was evaluated, a change in structure was visible before and after exposure to an electric current (FIGS. 4 and 5, respectively).

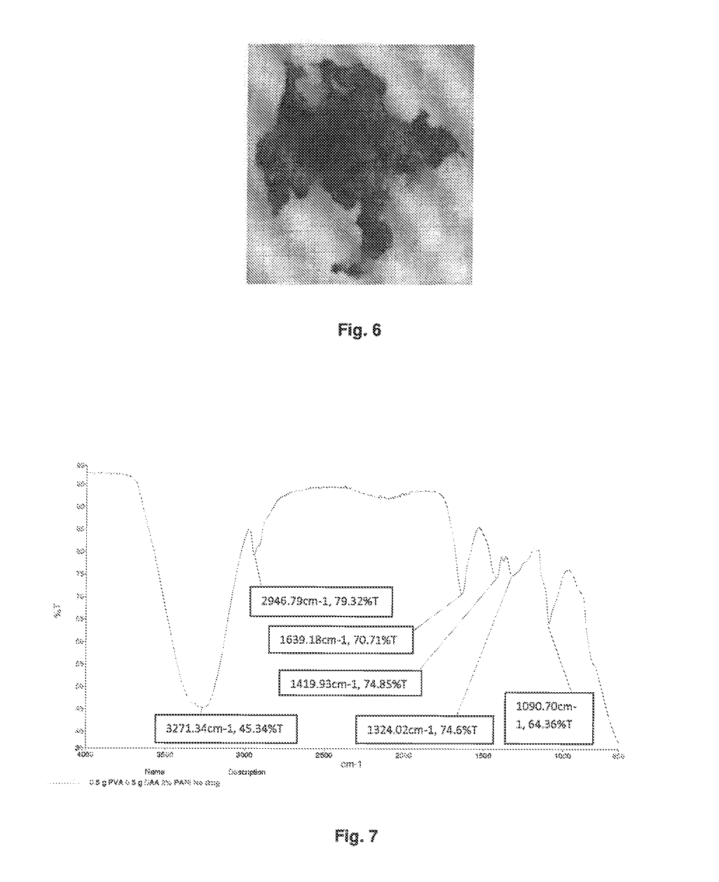

The hydrogel system in FIG. 5 has erosions on the bottom, which was the side exposed to the electrode. Therefore, it is possible to assume that the release mechanism may be due to the erosion of the hydrogel, thus resulting in liberation of the drugs. When the same hydrogel was made without the PANi, erosion did not occur, suggesting that PANi is somehow related to the erosion of this hydrogel. FIG. 6 shows the hydrogel system after 60 samples had been taken, clearly depicting the erosion which occurred on the hydrogel system when exposed to electric current. This erosion on the hydrogel was a surface phenomenon only.

Spherical erosions can be seen at sites where the electrodes had been placed on the hydrogel. The colour of the hydrogel became lighter in places where PANi was now absent, appearing as translucent areas on the hydrogel in FIG. 6. Drug release studies beyond 70 samples showed that even though drug release was no longer occurring, the hydrogel system was still undergoing erosion. This suggested that indomethacin does not partake in the erosion of the hydrogel.

The FTIR Spectroscopy of the PANi-Hydrogel System with and without Indomethacin

The applicant also investigated whether any reaction occurred between the hydrogel and the indomethacin. This is important from a release mechanism point of view because if indomethacin does have any interaction with the hydrogel system, there is a possibility that indomethacin may affect the structural integrity of the hydrogel and therefore the erosion rate. This would ultimately affect the release rate of indomethacin from the hydrogel system. In order to determine if there was any reaction between the indomethacin and the PANihydrogel system, Fourier Transform Infra-Red (FTIR) was performed using a Spectrum 100 (Perkin Elmer, Waltham, Mass., USA). The experiment was conducted in order to assess for any structural changes in a hydrogel system which was loaded with indomethacin compared to the same hydrogel system without indomethacin.

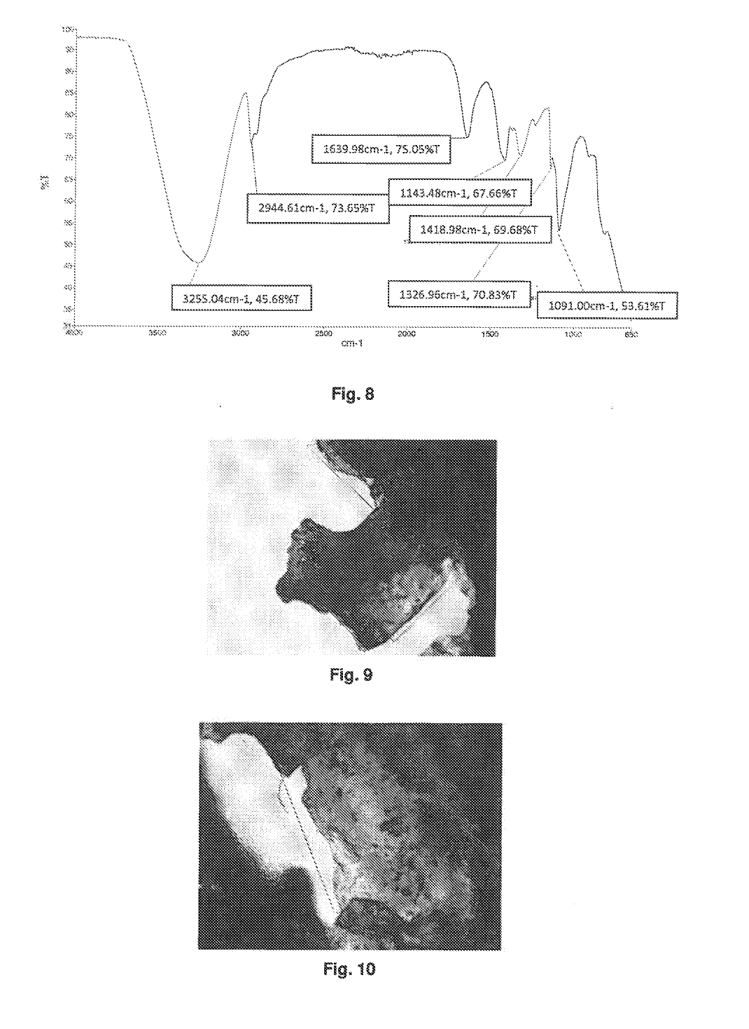

As shown in FIGS. 7 and 8, there is no difference in the hydrogel system which was loaded with indomethacin compared to the same hydrogel system without indomethacin, and therefore indomethacin does not have any direct interactions with the hydrogel system. This suggests that the mechanism whereby the drug is merely entrapped in the hydrogel system is in the form of nano-spheres and is liberated when the hydrogel system undergoes erosion, i.e. the drug is trapped in the hydrogel system during the crosslinking process and remains within the hydrogel system even during the swelled state until erosion occurs. There is no interaction between the drug and the hydrogel system.

Light Microscopy of the Eroded PANi-Hydrogel System

The surface morphology was analysed to see if there were any differences between the hydrogel system and the erosion sites, thus determining the possible causes of the erosion.

FIGS. 9 and 10 show the surface morphology of two different erosion sites captured on indomethacin-loaded hydrogels when using an Olympus SZX7 ILLD2-200 light microscope (Olympus, Tokyo, Japan).

The hydrogel at the erosion site was lighter than other areas. This may be attributed to the decrease in PANi as erosion takes place, since it is the PANi that gives this hydrogel system its distinctive black colour. It was therefore possible to link PANi to the erosions which occur at these sites. As previous experimentation has shown, the hydrogel system which was formed without PANi did not undergo any erosion when exposed to electric current, strongly suggesting that the attraction of PANi towards the gold counter electrode plays an important role in the erosion of the hydrogel system.

Electron Microscopy of the Eroded PANi-Hydrogel System

Scanning electron microscopy (SEM) was used in order to examine the surface morphology of the erosion site at 300-400× magnification. A Phenom™ (FEI Company, Hillsboro, Oreg., USA) SEM was used.

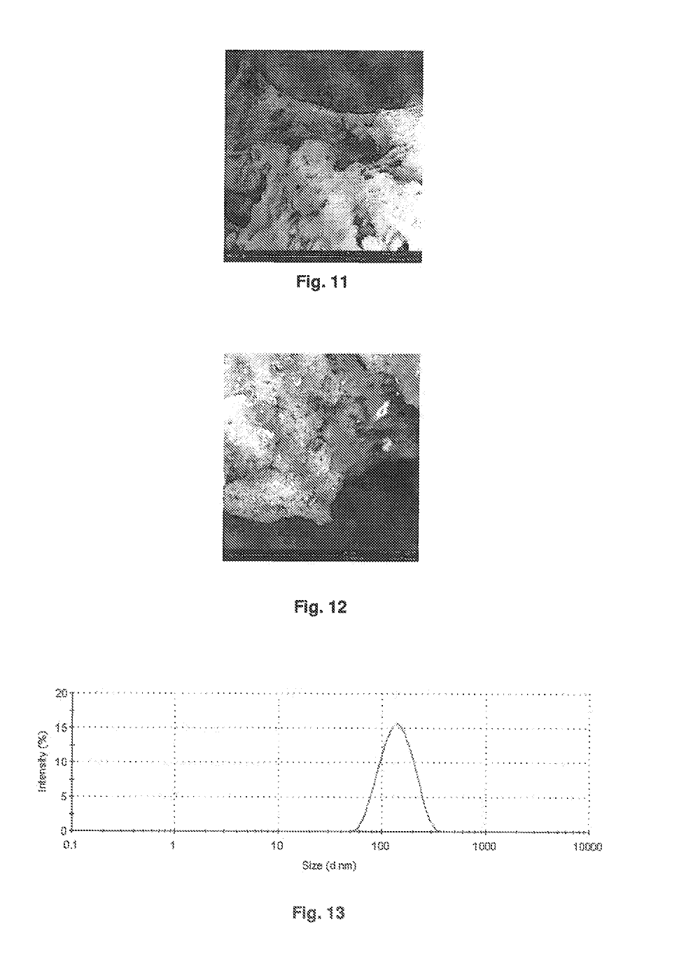

FIGS. 11 and 12 show the difference in surface morphology of two hydrogels. The uneroded hydrogel system exhibited a smooth surface morphology, which became a rough surface after the erosion had occurred. This may be due to the breaking of the crosslinked hydrogel structure, as pieces of the hydrogel system break away from the main hydrogel, leaving the surface irregular and with a rough texture.

Determination for Presence of Nano-Spheres by Dynamic Light Scattering

The presence of any nano-spheres in the hydrogel was determined via light scattering at 3TC at varying angles. The equipment used for this technique was the Zetasizer NanoZS (Malvern Instruments Ltd, Malvern, Worcestershire, UK). The hydrogel was formulated, cut in half and immersed in distilled water for 24 hours to allow adequate diffusion of nanosphere from the hydrogel system into the distilled water. Samples were then taken from the hydrogel-immersed distilled water and analyzed with the ZetaSizer NanoZS. The results indicated that nano-spheres were present, with a size range of approximately 138 nm (FIG. 13).

Determination of PVA and DAA on the Rate of Erosion of Hydrogels in the Presence of Electric Current

The effects that PVA and DAA have on the erosion of the hydrogel system were determined. For this experiment, 5 hydrogel systems with varying constituents were synthesized and exposed to an electric current. Each hydrogel contained 100 mg indomethacin and 2% w/w PANi, with varying amounts of PVA and DAA. The 5 hydrogel systems which were synthesized are shown in Table 4.

TABLE 4
Quantity of DAA and PVA used for the synthesis of each hydrogel

| | Hydrogel | Hydrogel | Hydrogel | Hydrogel | Hydrogel |
|---|---|---|---|---|---|
| | 1 | 2 | 3 | 4 | 5 |
| DAA | 0 g | 1 g | 0.5 g | 0.5 g | 0.25 g |
| PVA | 0.5 g | 0.5 g | 0.25 g | 1 g | 0.5 g |

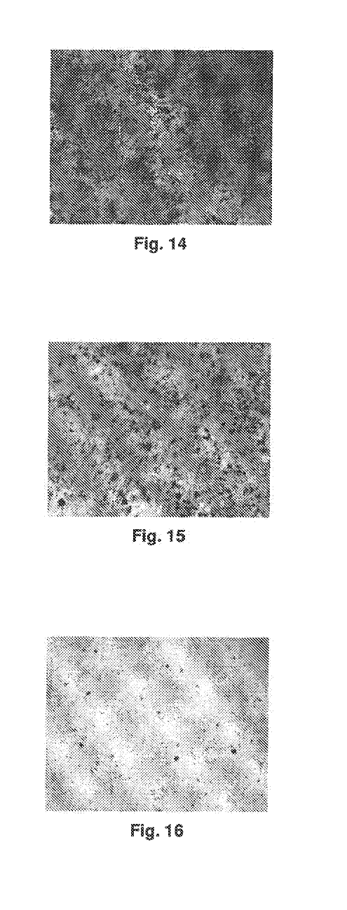

Each of the devices were then immersed in 25 mL of PBS and exposed to 1.2 V of potential difference for 10 minutes. The devices were then assessed for the extent of erosion and hence the effect which PVA and DAA have on the hydrogel system. Hydrogel 1 had the highest erosion rate, whereas hydrogels 2, 3 and 4 exhibited only a minimal erosion rate, with hydrogel 2 having the lowest erosion rate. Hydrogel 5 had a considerable erosion rate compared to hydrogels 2, 3 and 4 but less than hydrogel 1. The results observed in Table 3 can be explained by the crosslinking mechanism between DAA and PVA. The erosion rate is dependent on two factors: the degree of crosslinking and the concentration of PANi in the hydrogel. The lesser the degree of crosslinking and the higher the concentration of PANi, the higher the rate of erosion is going to be. In hydrogel 1, DAA was not present, which decreased the degree of crosslinking between the PVA. Since there was no DAA, the volume of the hydrogel was smaller, and thus the concentration of PANi was higher and the rate of erosion was the highest. In hydrogel 2, the amount of DAA was twice that of the PVA and the volume of the hydrogel was three times that of hydrogel 1. Therefore, the concentration of the PANi in the hydrogel system was decreased and the erosion rate was the lowest. Hydrogel 3 also included DAA, but in a smaller volume compared to hydrogel 2, and therefore had a higher degree of crosslinking and a higher concentration of PANi. The erosion rate was thus minimal but still higher than that of hydrogel 3. Hydrogel 4 was the opposite of hydrogel 3. In this hydrogel, the PVA was much higher than the DAA, therefore reducing the degree of crosslinking between the two. However, the volume of the entire hydrogel was equivalent to hydrogel 2, thus lowering the concentration of PANi in the hydrogel system. This lowered the erosion rate of the system. Hydrogel 5 showed a higher erosion rate than hydrogels 2, 3 and 4 because the PVA was dominant over DAA, thus lowering the degree of crosslinking as compared to hydrogel 3. The volume of this hydrogel system was also half of that of hydrogels 2 and 4. The concentration of PANi, however, was not higher than that that of hydrogel 1, and therefore, although it exhibited a higher erosion rate when compared to hydrogels 2, 3 and 4, it was still lower than that of hydrogel 1. In order to demonstrate the effect that volume has on the concentration of PANi, the hydrogel systems with various volumes of PANi were observed using a light microscope. In this experiment, only the amount of PVA was varied, while the rest of the constituents were kept at a constant 0.5 g DAA, 100 mg indomethacin and 2% w/w PANi. These hydrogels are shown in FIGS. 14-16. FIG. 14 depicts a hydrogel with 0.25 g PVA, which had the smallest volume. FIG. 15 shows the hydrogel with 0.5 g PVA, which had an intermediate volume. FIG. 16 shows the hydrogel with 1 g PVA, which had the largest volume.

FIGS. 14-16 show that with an increase in volume of the hydrogel system, there is a decrease in concentration of the PANi, as indicated by a decrease in distribution of the black particles. As the volume of the hydrogel gets bigger, the more spread out the PANi becomes, and thus the less electro-responsive the hydrogel becomes. In order to further substantiate the effects that PANi concentration has on the erosion rate of the hydrogel system, two separate hydrogel systems were formulated, each with 0.5 g PVA, 0.5 g DAA and 100 mg indomethacin. The only difference was that the first hydrogel system included only 1% w/w PANi while the second hydrogel included 3% w/w PANi. The two hydrogels were then immersed in 25 mL PBS and a potential difference of 1.2V was applied for 400 seconds in order to assess the erosion rate. As speculated, the hydrogel system with the 3% w/w PANi exhibited a significantly higher erosion rate than that of the 1% w/w hydrogel system. It is therefore important to bear in mind the PANi concentration of the hydrogel system when formulating the drug delivery system.

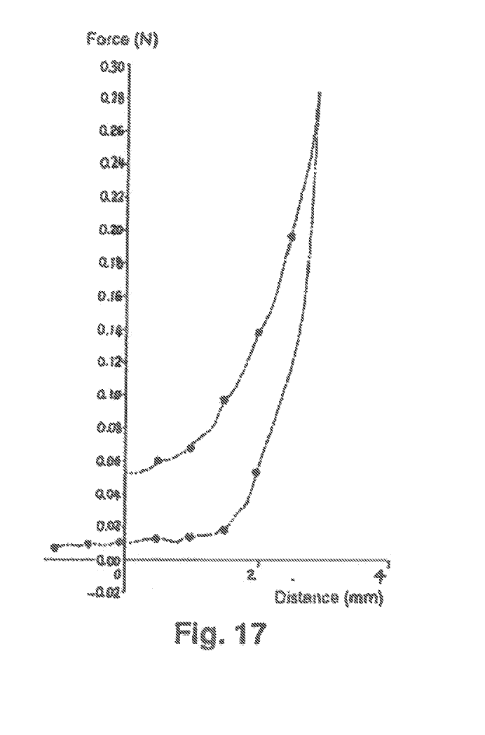

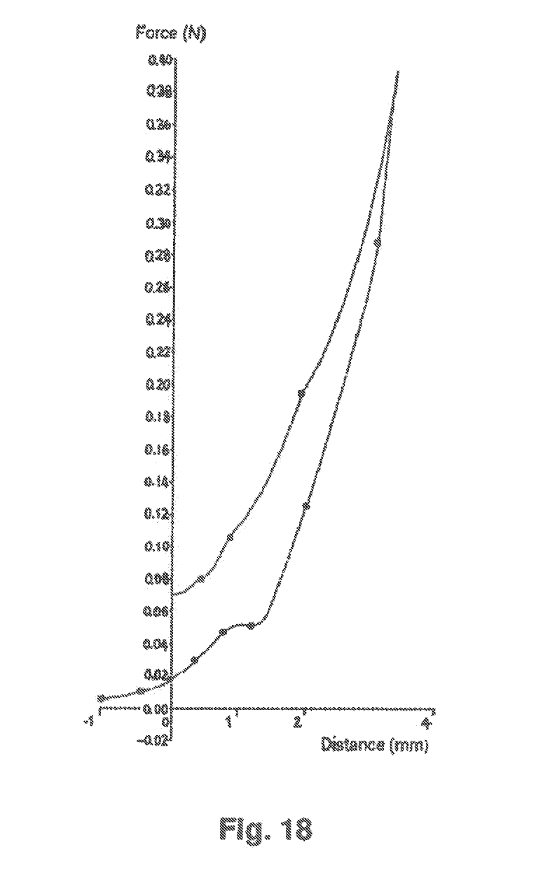

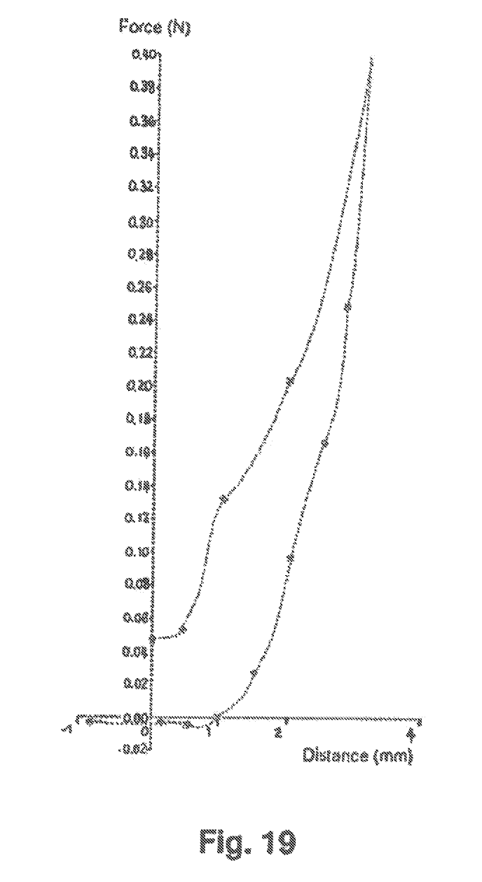

Another important factor which appeared to determine the erosion rate was the amount of DAA added into the system. The more DAA that was added into the system, the less the rate of erosion This suggested that DAA plays a role in hindering erosion rate, possibly due to the increased crosslinking within the hydrogel system. In order to confirm this, texture analysis was conducted on 3 different hydrogels using a gel compression test. All 3 hydrogels were composed of 2% w/w PANi, 0.5 g PVA and 100 mg indomethacin, with the difference being that the amount of DAA used was 0 g, 0.25 g and 1 g. The hydrogels were compressed to a distance of 3 mm, with a compression rate of 1 mm/second. The force required to compress each hydrogel over a distance of 3 mm was then recorded and is presented in FIGS. 17-19.

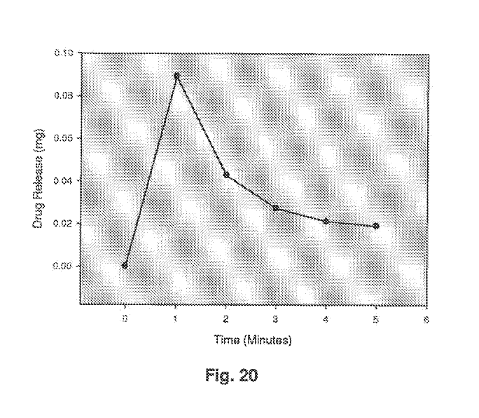

The results show that there is an increase in the required force to compress the hydrogel by 3 mm when DAA is incorporated into the hydrogel system. The required force for compression is the same for 0.25 g DAA and 1 g DAA, indicating there is an upper limit to the crosslink between PVA and DAA. This increase in force for compression when DAA is added may therefore indicate a crosslink between the DAA and the PVA as opposed to PVA alone. This crosslinked system was also tested by formulating two hydrogel systems, one with DAA and one without DAA. The two hydrogel systems were then assessed for their drug release capability in the presence and absence of electric current. The two hydrogels were immersed in 20 mL of PBS and a potential difference of 1.2V was applied for duration of 5 minutes. 4 mL samples were taken afterwards and assessed for drug release. The PBS was then discarded and the hydrogel systems were immersed in a fresh batch of 20 mL PBS. Samples were taken from 5 different hydrogel systems. FIG. 20 depicts the drug release from a hydrogel system without DAA and FIG. 20 depicts the drug release from a hydrogel system with DAA.

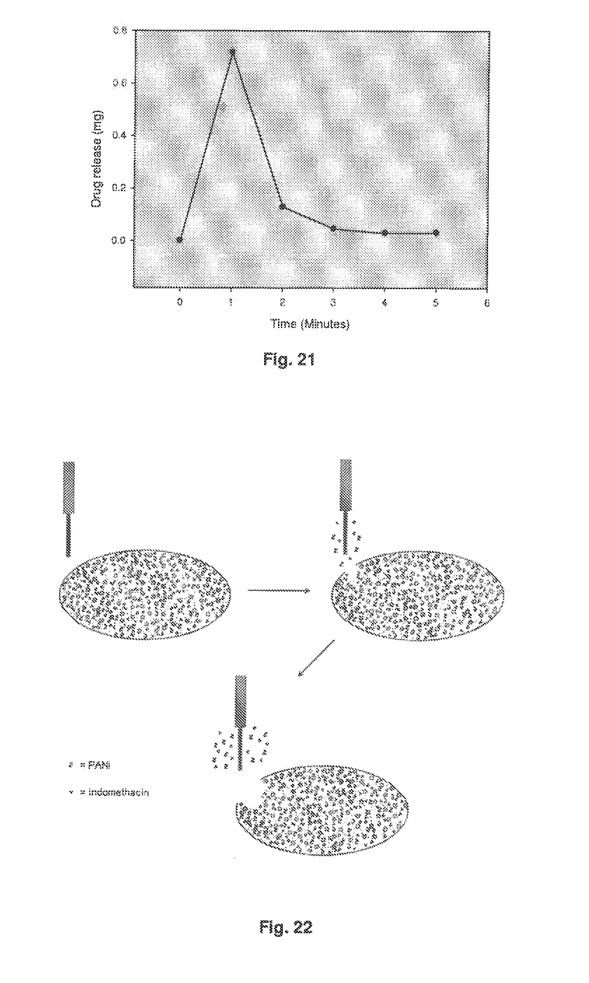

From FIGS. 20 and 21, it can be seen that the drug release drops significantly with the addition of DAA, thus suggesting the role of DAA in the hydrogel system as a crosslinker. Drug release is the highest at 5 minutes and drops gradually from 10 minutes onwards. When the hydrogel system was placed under the two electrodes during the drug release study, PANi was seen coating and floating around the gold counter electrode. Therefore, it was concluded that PANi was drawn towards the gold counter electrode. Experiments have shown that when PANi was incorporated into the crosslinked hydrogel system, it decreased the degree of crosslinking by becoming entrapped between the three dimensional network of the hydrogel system. When the gold counter electrode was placed onto the surface of the hydrogel, the PANi which was entrapped became drawn to the electrodes, and released itself from the hydrogel system. The PANi may break the crosslinked bond between the PVA and the DAA during this process, thus resulting in a weakening of structure and ultimately erosion. Since only the PANi which is close to the gold counter electrodes is drawn, only the structures around the electrodes will be weakened, thus explaining the phenomenon of surface erosion. This is represented by FIG. 22.

This mechanism of erosion would require an even and adequate distribution of PANi throughout the hydrogel in order to achieve optimum drug release. As seen in FIG. 9, the opaque areas where PANi was depleted ceased to erode in the presence of the electric current.

Using UV-visible spectroscopy, it was seen that the drug release was enhanced when electric current was passed through the PBS in which the polymeric hydrogel was immersed. The actual mechanism of this enhanced release is attributed to the erosion which causes the drug to be released into the surrounding medium. In contrast to the control, the experiment had a pulse release, as opposed to a first order release from that of the control.

Conclusions

Although the lack of mechanical strength and weak physical property may be a drawback to the hydrogel, it is possible to create an electroactive polymer hydrogel composition for use as an implantable drug delivery system by incorporating different hydrogel polymers, electroactive polymers and drugs.

### Example 2

### Preparation of the Cross-Linked Polyvinyl Alcohol Stimuli-Actuated Polymeric Device (SAPD) with Indomethacin as a Model Drug

Four different devices were constructed and loaded with indomethacin in order to assess its electroactivity. The compositions of the four devices are described hereunder.

The method of preparation for the first device was different than that of the other three devices and is discussed separately. In these devices, the PVA formed the hydrogel component, PANi acted as the EAP, Eudragit formed the crosslinker and indomethacin was the drug intended for targeted delivery.

Reagents for the PVA/PANi-Based SAPD

Device 1
PVA 8/88 (10 g)
Aniline (5 g)
Ammonium Persulfate (6.126 g)
Indomethacin (100 mg)

Device 2
PVA 8/88 (5 g)
PANi emeraldine doped (1.5 g)
EudragitR RL 100 (5 g)
PEG 4000 (5 g)
Indomethacin (100 mg)

Device 3
PVA 8/88 (5 g)
PANi emeraldine doped (1.5 g)
EudragitR L100-55 (5 g)
Indomethacin (100 mg)

Device 4
PVA 8/88 (5 g)
EudragitR L 100-55 (5 g)
PEG 4000 (5 g)
PANi emeraldine doped (1.5 g)
Indomethacin (100 mg)

The Synthesis of Drug-Loaded Stimuli-Actuated Polymeric Device

Device 1 was prepared by dissolving PVA (10 g) into 100 mL of 1.0M HCl acid. Aniline (5 g) was added into a 50 mL of 3.0M HCl acid and stirred until fully dissolved and added into the polyvinyl alcohol solution. This mixture was left to cool in an ice bath. Ammonium persulfate (6.126 g) was dissolved in another 50 mL of 3.0M HCl acid separately and added drop-wise into the PVA/Aniline mixture for a time period of one hour whilst being stirred vigorously in an ice bath. This was to ensure that the polymerization of aniline was carried out under cold conditions.

The ammonium persulfate acts as an oxidizing agent and an initiator to the polymerization of aniline. Following the addition of ammonium persulfate, the suspension was left to stir in an ice bath over a period of five hours before it was left in the fridge overnight for the polymerization to complete. The suspension was placed under a fume cupboard for 96 hours at room temperature in order to achieve maximum evaporation of the solvent. It was immersed in a 1:1 solution of acetone and 1.0M HCl solution for 12 hours to wash away all unreacted monomers. The hydrogel was dried for another 24 hours to ensure further evaporation of solvents. In order to load drugs into the system, 100 mg indomethacin was dissolved in 100 mL of heated PBS, the hydrogel was dissolved in 100 mL heated distilled water and the two solutions were mixed together. Since PBS is miscible with water, it would ensure a homogenous mixture of the drug into the PVA hydrogel. The resulting drug-loaded PVA hydrogel was then left to stand in a mould until solidified.

The preparation for Device 2, 3 and 4 were identical, except for variation of the ingredients used (see above). PVA (5 g) was dissolved in 20 mL of boiling water followed by cooling to room temperature. Eudragit (5 g) and PEG 4000 (5 g) were dissolved in two separate beakers with 20 mL dichloromethane and mixed together once both were fully dissolved.

Indomethacin (100 mg) was added into the EudragitR/PEG 4000 mixture and stirred until dissolved followed by addition into the cooled PVA solution. Due to the immiscibility between dichloromethane and water, the volume of water was kept to a minimum of 20 mL. The resulting emulsion was blended with a pestle and mortar for ten minutes, followed by homogenizer for one minute. The emulsion was poured in a mould and left to dry in a fume cupboard for a period of 96 hours to ensure evaporation of the solvent. Once dried, the SAPD was rinsed with distilled water to ensure removal of any drug not incorporated into the hydrogel.

Gel Compression Measurement of the Stimuli-Actuated Polymeric Device

After the SAPD was synthesized, the hydrogels were tested for their strength to withstand forces. This was important as gel compression allowed us to assess the degree of crosslinking in the hydrogel. Gel strength was assessed with the use of a texture analyzer.

The SAPDs were weighed and placed onto the TA.XT.plus Texture Analyzer (United Scientific, Gauteng, South Africa) where it was compressed by the rounded end probe. In this test, the rate of compression and time was set as a constant, while the force required for such compression was measured. The Texture Analyzer was set to move at a rate of 1.535 mm/sec for a period of 2.890 seconds. The total compression into the hydrogel is depicted as Total Compression=Rate of movement×Time. Accordingly, the total compression into the hydrogel was 4.436 mm. The force required for compression of each device as measured by the texture analyzer:

Device 1—0.9969 N

Device 2—1.3011 N

Device 3—0.9750 N

Device 4—0.0011 N

The most force was required for the compression of Device 2. This may indicate an increased crosslinking between PVA and EudragitR RL when compared to Device 1 and 3, while Device 4 required the least force for compression. This indicated that Device 4 may have to be handled with care due to its weak structural integrity and may not be favoured as a pharmaceutical formulation. All hydrogels were capable of returning to its original shape after compression which suggested that the yield value for these hydrogel may be much higher.

Surface Morphology of the Stimuli-Actuated Polymeric Device

The next part was to assess the surface morphology of the SAPD. The surface morphology was closely linked to the texture and also gave an indication of the drug release profile of the SAPD. A porous hydrogel should release drug easier as opposed to a hydrogel which possessed a non-porous structure. The surface morphology of the SAPD was determined by using light microscopy. In addition, a porous hydrogel would probably be preferred due to its increased ability to absorb solvents and faster drug release profile as opposed to non-porous hydrogels.

Device 2, 3 and 4 have shown enhanced porosity in comparison to Device 1. This indicated that Device 2 may be the favoured Device, due to its porous structure and its ability to withstand high stress. Although Device 1 and Device 3 have both shown promising ability to withstand stress, Device 1 possessed a smooth surface morphology and therefore drug release from Device 1 may be hindered. Thus far, Device 2 and 3 seemed to be the most promising for the purpose of drug delivery.

Setup of the Circuit for In Vitro Assessment of Drug Release from the Stimuli-Actuated Polymeric Device in the Presence of an Electric Current

In order to determine the drug release profile of the drug-loaded SAPD, the devices were immersed into 100 mL of PBS and electric currents were allowed to pass through it. The source of the current was obtained from a 9V battery cell. Copper wires 20 cm in total length with a diameter of 2 mm were used as the conductor and the electrodes used were iron electrodes. A multi-meter was connected in series in order to assess the amount of current passing through the circuit at any given time.

The devices were placed into 100 mL of PBS and 9V of potential difference was applied to the circuit. The reading on the multi-meter indicated that 30 mA of currents were passing through the circuit at the time of testing. PBS samples of 5 mL were drawn via a syringe and replaced with 5 mL of fresh PBS at various time intervals. The samples were analyzed for any presence of drugs in order to assess the behaviour of drug of the device. The time intervals for this study were 1, 2, 3, 4 and 5 minutes. These samples were tested for drug concentration via the use of an UV-visible spectroscopy. For comparison purpose, the device was divided into two pieces and one half was placed in 100 mL of PBS in the absence of electric current to serve as control. Samples in the control group were collected at the same time intervals and assessed for any difference in drug concentration than that of the experiment.

The Drug Entrapment Efficiency of the Stimuli-Actuated Polymeric Device

In order to test the drug entrapment of these devices, Device 4 was duplicated and loaded with 120 mg of indomethacin. The Device weighed a total of 4.295 g. This test was done in triplicate in order to ensure a consistent result. For the first test, a portion of the device with a mass of approximately 22 mg was removed and dissolved in 80 mL of heated PBS with the aid of a homogenizer. A sample of 1 mL was taken from the 80 mL and diluted with 4 mL PBS.

Using 0.01=9.7735x one can solve for x as being 0.0010 mg/mL in the 1:4 diluted samples. Multiplying by 5 provides the amount of drug in 1 mL of the sample, and further multiplication by 80 provides the total amount of drug in 22 mg sample which was dissolved in 80 mL PBS.

The total amount of drug present in the entire device equals 79.85 mg and the total drug which was entrapped equals 66.54%

For the second test, Device 4 was duplicated, with a total weight of 8.565 g and loaded with 200 mg indomethacin. 0.430 g of sample was removed and dissolved in 100 mL PBS, after which 1 mL was diluted with 4 mL of PBS again and scanned under the UV-visible spectroscopy. The reading obtained was 0.121 A. The total amount of drug present equated to 6.2 mg indomethacin in the 0.430 g sample. Therefore, the drug entrapment efficiency was 61.74%.

For the third test, Device 4 was duplicated and loaded with 200 mg of indomethacin. The total weight of the device was 7.654 g and a 0.385 g sample was removed and assessed. The sample was dissolved in 100 mL PBS, after which 1 mL sample was extracted and diluted with 4 mL PBS. The results obtained from the UV/visible spectroscopy indicated an absorbance of 0.145 A. Therefore, the total amount of drug in the 0.385 g sample was 7.418 mg. This equated to 147.474 g of indomethacin in the Device and a drug entrapment efficiency of 73.737%

From the above three tests, the drug entrapment efficiency of the device fell approximately in the range of 60-70% during synthesis.

The Amount of Drug Release from Stimuli-Actuated Polymeric Device Under the Influence of Electrical Current

The samples were previously evaluated for any presence of drug by employing the UV visible spectroscopy. The results obtained for both experimental and control Devices 1-4 is shown in Tables 5-8. The results are obtained in UV absorbance at a wavelength of 318 nm. Study done on PANi has indicated that PANi has an absorbance peak at 365 nm, 460 nm and over 820 nm.

TABLE 5
The UV-absorbance of samples collected from Device 1.

| Device 1 | 1 minute | 2 minutes | 3 minutes | 4 minutes | 5 minutes |
|---|---|---|---|---|---|
| Experiment | 0.0001 mg | 0.0001 mg | 0.0002 mg | 0.0002 mg | 0.0002 mg |
| (4.637 g) | | | | | |
| Control | 0.0001 mg | 0.0001 mg | 0.0001 mg | 0.0002 mg | 0.0002 mg |
| (4.204 g) | | | | | |

There was no drug release from both the experiment and the control. There was negligible difference with regards to the amount of drug present in the sample and this device was not electroactive.

TABLE 6
The UV-absorbance of samples collected from Device 2.

| Device 2 | 1 minute | 2 minutes | 3 minutes | 4 minutes | 5 minutes |
|---|---|---|---|---|---|
| Experiment | 0.055 mg | 0.054 mg | 0.041 mg | 0.044 mg | 0.046 mg |
| Control | 0.015 mg | 0.016 mg | 0.019 mg | 0.015 mg | 0.020 mg |

The results obtained from Device 2 have indicated an enhanced release of indomethacin in the presence of electric current, as opposed to the control, which had a decreased rate of release. The slight decrease in drug concentration between various time intervals may be due to the dilution of the conducting medium when 5 mL of PBS was used to replace the samples that were taken. These values were transformed into the quantity of drug present in the sample and would be discussed further on.

TABLE 7
The UV-absorbance of samples collected from Device 3.

| Device 3 | 1 minute | 2 minutes | 3 minutes | 4 minutes | 5 minutes |
|---|---|---|---|---|---|
| Experiment | 0.213 mg | 0.233 mg | 0.258 mg | 0.280 mg | 0.337 mg |
| Control | 0.106 mg | 0.100 mg | 0.102 mg | 0.129 mg | 0.219 mg |

Results from Device 3 have also indicated an enhanced release of indomethacin in the presence of electrical stimulation.

TABLE 8
The UV-absorbance of samples collected from Device 4.

| Device 4 | 1 minute | 2 minutes | 3 minutes | 4 minutes | 5 minutes |
|---|---|---|---|---|---|
| Experiment | 0.051 mg | 0.111 mg | 0.084 mg | 0.082 mg | 0.115 mg |
| Control | 0 mg | 0 mg | 0 mg | 0 mg | 0 mg |

Device 4 successfully released drug only in the presence of electrical current, while withholding the drug in the absence of electrical stimulation. Device 4 seemed the most promising following the assessment of the drug release profile in the presence of an electrical field. However, the weak structural integrity meant that it should be handled with caution. It was also composed of a porous structure which indicated that it was capable of fluid absorption. The initial drug release study has shown that Device 4 may have the optimum drug release profile, however, further studies should be performed in order to assess the consistency and the mechanism of drug release of Device 4.

Concluding Remarks

This study has shown that the use of emeraldine based PANi as an electro-responsive polymer in a crosslinked hydrogel may be used for the controlled release of a drug, for example, indomethacin. The degree of response from these polymers may vary depending on the conditions of synthesis of these polymers.

EAPs incorporated into a crosslinked hydrogel have shown electrical conductivity, and the release of indomethacin was enhanced when indomethacin was blended into the hydrogel with an EAP in the presence of an electric current in the present study. Hydrogel systems which were undoped did show drug release even without the presence of an electrical current, although the degree of drug release was to a much smaller extent. The crosslinking of the hydrogel did successfully withhold the drug within the hydrogel, but such crosslinking also retarded the extent of drug release in the presence of electrical stimulation. This release may be due to the repulsive force caused by the like-charges of the negatively charged electrons from the electric current and the negatively charged (anionic) drug indomethacin, or it may be linked to processes such as iontophoresis. The use of PANi along with a crosslinked hydrogel may be a prospective drug delivery device for the controlled release of indomethacin by the means of an electrically-activated and controlled device in patients. Aniline, which underwent polymerization in the solution of polyvinyl alcohol in 1.0M HCl acid showed no response when placed in electric current for the purpose of controlled release of indomethacin. However, PANi emeraldine which was incorporated into the polymeric hydrogel device did indeed demonstrated enhanced release of indomethacin. These release rates have shown to increase linearly as time progresses. In order for these devices to become practical, characteristics such as increased drug release should be investigated further. In addition, the response time should not exceed 1-2 minutes. This could be achieved by increasing the surface area of the hydrogel which would allow more drugs to move towards the charged electrodes.

Animal Studies

The hydrogel composition, herein after referred to as the SAPD, as per Example 1 above was used in the animal studies.

Materials and Methods

Mobile phase used was acetonitrile (Merck, Wadeville, Gauteng, South Africa), orthophosphoric acid (Merck, Wadeville, Gauteng, South Africa) and double deionised water obtained from the Milli-Q System. Control blank rat plasma was supplied from healthy donor. The drug used was indomethacin (Sigma Aldrich, Steinhelm, Germany)). Healthy Sprague-Dawly rats were used for this in vivo release study. Blood samples were analyzed with the High Performance Liquid Chromatography (HPLC) model Waters 1525 Binary Pump with 2489 UV/visible detector. The column was a C18 silica gel column (4.6_{—}150 mm, 5 μm particle size, Waters) while the pre-filter used was a 0.44 μm MilliporeR filter. Further analysis was done with the Ultra Performance Liquid Chromatography model Waters Acquity Ultra Performance Liquid Chromatography System (Waters, Milford, Mass., USA). The column used in the UPLC was a C18 column (2.1_{—}50 mm, 1.7 μm particle size, Waters). Tissue samples were sent to the IDEXX Laboratories for histological analysis.

In Vivo Studies to Assess the Biocompatibility and Drug Release Kinetics from the Stimuli-Actuated Polymeric Device

Studies were conducted on Sprague-Dawley rats in order to assess the drug release profile of the SAPD. The SAPD was implanted subcutaneously, blood samples were taken at predetermined time intervals and the results of the Control Group were compared to that of the Placebo Group. This would allow the quantification of the drug release from the SAPD in vivo in the rat. The blood samples were assessed for any presence of drug which would indicate drug release from the SAPD. In addition, tissue samples were harvested from the implantation site and histological examinations were performed in order to assess for any long-term inflammations or tumor formations.

Method and Approach for the In Vivo Study of the Stimuli-Actuated Polymeric Device

All animal study procedures and surgeries were performed in collaboration with the Central Animal Service (CAS) of the University of Witwatersrand. The number of animals required for this study was 18 and included both males and females having a body mass of between 200-250 g.

This study was an interventional study. 18 rats with an initial weight of 200-250 g were randomly assigned to 3 groups (n=6 in each group).

1. Test group 1 (n=6): SAPD was subcutaneously implanted into the flank (abdominal area) of each animal in this group. This group received a SAPD containing approximately 16 mg/kg of indomethacin.

2. Placebo group 2 (n=6): SAPD was subcutaneously implanted into the flank (abdominal area) of each animal in this group. This group received a drug-free SAPD.

3. Comparison group 3 (n=6): The rats in this group would receive intravenous administration of indomethacin (0.8 mg/100 g body weight) 15 minutes prior to their blood sample taken.

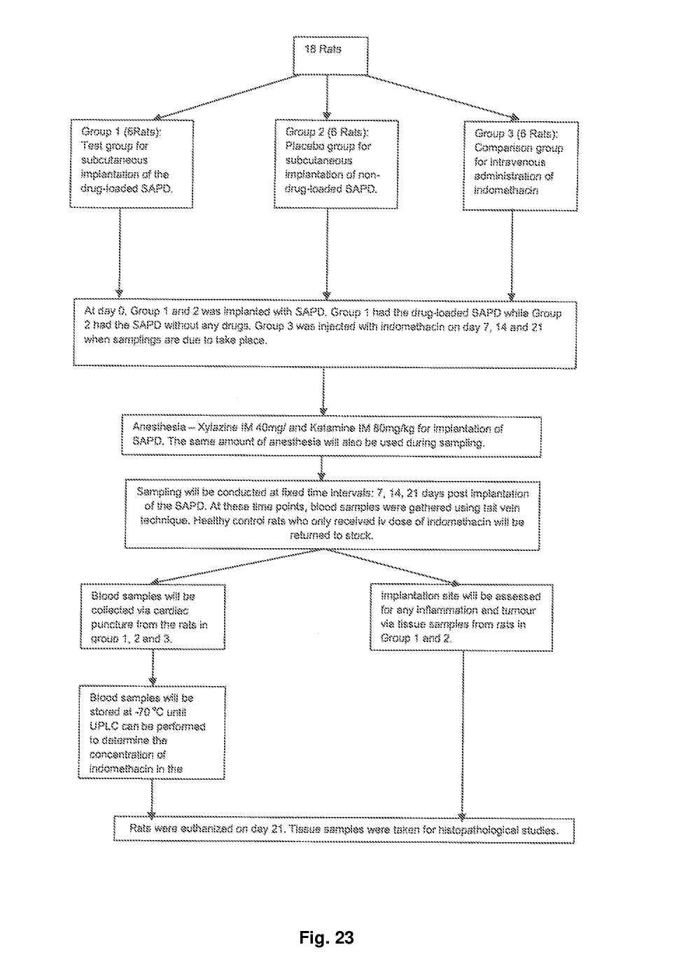

All the groups were provided with water and food ad libitum. The rats was caged in groups of fifteen and maintained on a 12 hour light/12 hour dark cycle. They were weighed daily so as to indicate their general state of well being. Cage activity by means of observation for 1 hour periods daily was in order to use to assess state of well being. At the final sampling point, rats from Group 1, 2 and 3 were sacrificed. The procedure of this animal study may be summarized by FIG. 23.

Implantation of the SADP

A SAPD (1×1×0.3 cm when fully hydrated) was implanted subcutaneously in the flank (abdominal area) into 6 Experimental Group rats and the 6 Placebo Group rats. This was performed while the rats were under anesthesia with xylazine (5 mg/kg) and ketamine (100 mg/kg). A 1.5 cm incision was made in the lower left flank for implantation and closed with a surgical wound clip. An injection of buprenophrine 0.1 mg/kg subcutaneous injection was administered for 3 days after the surgery.

The rats may develop an inflammatory response due to the presence of the implant. This would be treated with appropriate anti-inflammatory agents.

Once the SAPD was implanted, a potential difference of 1V would be applied to the SAPD implantation site. The duration of the electric current during the sampling was 1.5 minutes. This sampling procedure would take place on day 7, 14 and 21. The animals were inspected around the implantation site for any signs of inflammation and/or infection on day 7, 14 and 21. This was essential in determining the biocompatibility of the SAPD.

Drugs and Medicinal Substances to be Used for this Animal Study

The drugs used for this study had to be dosed according to the amount required for therapeutic effects in a rat model. This included any drugs used for anaesthesia, the model drug used in the SAPD and the drug used for euthanasia. These drugs were administered by a veterinarian at the Central Animal Service (CAS) of the University of Witwatersrand. These drugs and their uses are summarized below.

Indomethacin—i.v. 0.8 mg/100 g—i.v. administration of the drug will be done weekly

Indomethacin—contained in the SAPD—16 mg/kg—A once-off implantation of the SAPD and weekly sampling

Xylazine—Intramuscular injection—5 mg·kg—administered before implantation of SAPD and prior to euthenasia

Ketamine—Intramuscular injection—100 mg/kg—administered before implantation of SAPD and prior to euthenasia

Sodium Pentobarbitone—Intracardiac injection—200 mg/kg—administered once-off for euthanasia.

Plasma Sampling from the Rat Model after the Experimental Procedure

Plasma levels of indomethacin were measured via blood samples obtained around the SAPD implantation site and were analyzed using the High-Performance Liquid Chromatography (HPLC). The blood sample were obtained by the tail vein technique. This was performed by two people. One person restrained the rats by the use of a tightly-fitting, homemade bag of towelling while exposing the tail. The tail was held with one hand while an incision is made with the other hand. The tail was gently stroked and a blood drop formed at the site of incision. The blood drop was collected for analysis. The tail vein technique was advantageous as the animal need not be anesthetized and it is not a terminal procedure. The animal was returned to CAS until the next sampling was due to take place. The blood sample was stored in heparinised tube and centrifuge at 3000 G for 15 minutes. The plasma sample was stored at −70° C. until HPLC analysis.

Liquid-Liquid Extraction Technique for the Separation of Drug Bound in Plasma

Once the blood samples were extracted from the rat model, the drug was separated from the plasma sample before it was available for analysis via the HPLC. In order to achieve this, liquid-liquid extraction technique was used. The liquid-liquid extraction technique was used in cases where a solution contained two or more solutes. The solutes may be separated from each other by making use of the individual solubility of each solute. By placing the solution in the extraction fluid, the solutes would separate out from the solution into the extraction fluid based on its affinity for the extraction fluid. The extraction fluid typically contained an organic and inorganic phase and allowed the separation of nonpolar solutes into the organic phase and the polar solutes into the inorganic phase. The two solutes may be extracted following the evaporation of the respective solvents.

By using this technique, it was possible to separate the drug from a blood sample collected from the rat model. The blood was centrifuged and the plasma was collected. The processed plasma sample may be mixed with another solvent in order to extract the drug out of the plasma. In the case of indomethacin, an organic solvent should be used since indomethacin exhibits high solubility in organic solvents as compared to other solvents.

Subcutaneous Implantation of the Device into the Rat

The first step of this in vivo study was the subcutaneous implantation of the device into the rats. Eighteen rats were used for this study, with 3 rats housed per cage. The rats were caged one week prior to implantation to allow time for acclimatization. They were also weighed and checked on a frequent basis to ensure their health was in good condition prior to the implantation. Rats 1-6 received an implant which contained the active drug indomethacin, while rats 7-12 received a placebo device which contained no drug. The rats were numbered by the use of a permanent marker on the tail and were labelled 1-18 numerically.

Following the acclimatization of the rats, the devices were implanted subcutaneously while under general anaesthesia. The pulse during this time was monitored by the senior veterinary nurse, while the implantation was performed by the veterinary surgeon. The entire procedure was simple and quick, with each implantation taking 5-10 minutes. The devices were disinfected with Hibiscrub prior to implantation.

Results and Discussion

High Performance Liquid Chromatographic Analysis of Plasma Sample for the Presence of Indomethacin

Following the implantation of the SAPD into the rat model, blood samples were drawn on day 7, 14 and 21. The next step was the isolation of indomethacin from the plasma sample and analysis of drug release by utilizing the HPLC. The blood samples were taken from the rats which would indicate drug release from the device at the implantation site. Some of the drugs from the implantation site would be absorbed into the blood and presence of drug in the blood sample would indicate a release of drug at local site. Interstitial fluids cannot be used as sample since interstitial fluids in general does not yield enough quantity for analysis. Before the blood sample may be processed, it was collected in a heparinised tube. The recommended dosage for heparin is 70 units per 10 mL. The blood samples gathered was 0.5 mL, which required approximately 4 units of heparin. This equated to 4 μL of the heparin for the half mL blood samples. The collected blood samples were centrifuged and the plasma was extracted by the use of a 1 mL syringe. The indomethacin was extracted from the plasma samples by the use of the liquid-liquid separation technique as mentioned. This was done by adding 100 μL of phosphate buffer and 2 mL of ethyl acetate into 400 μL plasma sample. The mixture was vortexed vigorously for 10 minutes. The organic layer was separated and ethyl acetate was left to evaporate. The residue was re-dissolved in 200 μL of the mobile phase and 50 μL was injected for analysis by the HPLC.

The indomethacin peak was absent in the Placebo Group, which was used as a comparison for the Test Group. This absence of peak confirmed the release of drug in the test group. One set of blood sample taken between the 7 day release cycles has confirmed that, as indicated by the in vitro test, there was no drug leakage during each actuation. These results may be compared against the blood samples obtained from the comparison group in order to confirm that the degradation of the SAPD did not produce any possible untoward reaction during the release.

Further Analysis of Blood Sample with the Use of Ultra Performance Liquid Chromatography

The presence of drug in the rat plasma sample was further ascertained use the use of Ultra

Performance Liquid Chromatography (UPLC). The UPLC chromatogram obtained for the rat blood sample in the test group.

Blood samples injected into the UPLC from the placebo group did not exhibit a peak at approximately 3.5 minutes of the run time. This peak was observed in the blood sample obtained from the Test Group and corresponded with the results obtained from the HPLC.

The use of the UPLC has also confirmed the peak of indomethacin in the rat blood sample at a higher absorbance and a shorter run time. The retention time for the indomethacin in the UPLC was 3.62 minutes.

Blood samples were gathered for duration of 21 days with a release cycle every 7 days. The drug release over these 3 cycles was determined by the amount of drug present in the blood.

Although the blood concentration of the drug did not necessarily represent that of the drugs present at the local site, it was an indication of the drug release which has occurred locally and entered the systemic circulation.

The drug release profile of the SAPD from the in vivo rat model. The release cycle over the 21 day period has indicated a fairly consistent release and a favourable drug release profile.

Concluding Remarks

The use of an in vivo study has allowed us to determine the biocompatibility and the drug release profile when the SAPD was implanted subcutaneously. The SAPD was implanted subcutaneously into the rat model, and found to release drugs in the presence of electrical stimulation. This was done by sampling blood obtained from the rats at weekly interval. The results obtained from the HPLC and UPLC have indicated the presence of minute concentrations of drug in the blood, which ascertained the release of indomethacin from the SAPD into the surrounding tissue.

Results were also compared against the control group and interval blood samples obtained has shown that leakage of the drug has not occurred. This result also corresponds with that obtained from the in vitro study. The drug release from the SAPD over the 3 release cycles also seemed to be consistent.

The site of implantation did not show any signs of swelling, which indicated the absence of inflammation or infection. The rats weighed during the implantation period have also shown a steady increase in body weight and growth. Histological results showed no tumor or signs of significant tissue.

### REFERENCES

1. P. Bawa, V. Pillay and Y. E. Choonara, Stimuli-responsive polymer and their applications in drug delivery, Biomedical Materials 4 (2009), pp. 1-15.
2. S. W. Kim, Temperature sensitive polymer for delivery of macromolecule drugs, Advanced Material in Biomaterial in Biomedical Engineering and Drug-Delivery Systems, pp. 126-133.
3. G. G. Wallace, M. Smyth and H. Zhao, Conducting electroactive polymer-based biosensors, Trend in Analytical Chemistry 18(4) (1999), pp. 245-251.
4. Y. Bar-Cohen, K. J. Kim, H. R. Choi and J. D. W. Madden, Electroactive polymers, Smart Materials and Structures 16 (2007), pp. 1-2.
5. A. Mirmohseni and G. G. Wallace, Preparation and characterization of processable electroactive polyaniline-poly vinyl alcohol composite, Polymer 44 (2003), pp. 3523-3528.
6. H. Kim, S. J. Park, S. Kim, N. G. Kim and S. J. Kim, Electroactive polymer hydrogel composed of poly acrylic acid and poly (vinyl sulphonic acid) copolymer for application of biomaterials, Synthetic Materials 155 (2004), pp. 674-676.
7. C. J. Small, C. O. Too and G. G. Wallace, Responsive conducting polymer-hydrogel composite, Polymer Gels and Network 5 (1997), pp. 251-265.
8. L. M. Lira and S. I. C. de Torresi, Conducting polymer-hydrogel composite for electrochemical release devices: synthesis and characterization of semi-interpenetrating polyaniline-polyacrylamide networks, Electrochemistry Communications 7(7) (2005), pp. 717-723.
9. S. Murdan, Electro-responsive drug delivery from hydrogel, Journal of Controlled Release 93 (2003), pp. 1-17.
10. M. R. Semenchuk and S. Sherman, Effectiveness of Tizanidine in neuropathic pain—An open label study, Journal of pain 1 (4) (2000), pp. 285-292
11. S. Barakzoy and A. H. Moss, Efficacy of the World Health Organisation Analgesic Ladder to treat pain in end-stage renal disease, Journal of the American Society of nephrology 17 (2006), pp. 3198-3203.
12. G. R. Palos, T. R. Mendoza, S. B. Cantor, L. A. Aday and C. S. Cleeland, Perception of analgesic use and side effect—what the public values in pain management, Journal of pain and symptom management 28(5) (2004), pp. 460-473.
13. S Prasad, S. Ewigman and J. Hickner, Acute gout: Oral steroid work as well as NSAIDs, Journal of Family Practice 57(10) (2008), pp. 655-657.
14. Pleuvry, 2005 B. J. Pleuvry, Factors affecting drug absorption and distribution, Anaesthesia and intensive care medicine 6(4) (2005), pp. 135-138.

1. A polymeric hydrogel composition for the delivery of a pharmaceutically active agent to a human or animal when an electrical current is applied to the composition, the composition comprising:
a polymer which forms a hydrogel;
an electroactive polymer; and
a pharmaceutically active agent;
wherein the pharmaceutically active agent is released from the hydrogel composition when an electrical stimulus is applied to the hydrogel composition.

2. The hydrogel composition according to claim 1, wherein the polymer which forms the hydrogel is poly vinyl alcohol (PVA).

3. The hydrogel composition according to claim 2, wherein the polymer which forms the hydrogel is cross-linked with a cross-linking agent.

4. The hydrogel composition according to claim 3, wherein the cross-linking agent is diethyl acetamidomalonate (DAA).

5. The hydrogel composition according to claim 1, wherein the electroactive polymer is selected from the group consisting of polyaniline, polypyrrole and polythiophene.

6. The hydrogel composition according to claim 5, wherein the electroactive polymer is polyaniline.

7. The hydrogel composition according to claim 1, wherein the pharmaceutically active agent is an analgesic.

8. The hydrogel composition according to claim 7, wherein the analgesic is a non-steroidal anti-inflammatory drug (NSAID).

9. The hydrogel composition according to claim 1, wherein the pharmaceutically active agent is indomethacin.

10. The hydrogel composition according to claim 1, which is for use in relieving chronic pain.

11. The hydrogel composition according to claim 1, wherein the pharmaceutically active agent ceases to be released when the electrical stimulus is no longer applied to the hydrogel composition.

12. The hydrogel composition according to claim 1, which is in an implantable form.

13. The hydrogel composition according to claim 1, which is biodegradable.

14. The hydrogel composition according to claim 1, which provides controlled and targeted delivery of the pharmaceutically active agent.

15. The hydrogel composition according to claim 1, wherein the electrical stimulus is an electric current which is applied for a time period of from about 1 second to about 5 seconds.

16. The hydrogel composition according to claim 1, wherein a potential difference which is applied is from about 0.3 volts to about 0.5 volts.

17. A method of preparing a hydrogel composition according to claim 1 which is capable of delivering a pharmaceutically active agent to a human or animal when an electrical stimulus is applied to the hydrogel composition, the method comprising the steps of:
mixing a polymer for forming a hydrogel, a cross-linking agent, an electroactive polymer and a pharmaceutically active agent; and
allowing a hydrogel to form which contains the electroactive agent and pharmaceutically active agent.

18. The method according to claim 17, wherein the polymer which forms the hydrogel is poly vinyl alcohol (PVA).

19. The method according to claim 18, wherein the polymer which forms the hydrogel is cross-linked with a cross-linking agent.

20. The method according to claim 19, wherein the cross-linking agent is diethyl acetamidomalonate (DAA).

21. The method according to claim 17, wherein the electroactive polymer is selected from the group consisting of polyaniline, polypyrrole and polythiophene.

22. The method according to claim 21, wherein the electroactive polymer is polyaniline.

23. The method according to claim 17, wherein the pharmaceutically active agent is an analgesic.

24. The method according to claim 23, wherein the analgesic is a non-steroidal antiinflammatory drug (NSAID).

25. The method according to claim 17, wherein the pharmaceutically active agent is indomethacin.

26. A method of treating chronic pain in a human or animal, the method comprising the steps of:
implanting a hydrogel composition according to claim 1 in the human or animal at a targeted site of delivery; and
applying an electrical stimulus to the hydrogel composition to release a dose of a pharmaceutically active agent from the hydrogel composition.

27. The hydrogel composition according to claim 3, wherein the cross-linking agent is Eudragit.

28. The hydrogel composition according to claim 5, wherein the polyaniline is emeraldine base polyaniline.

29. The hydrogel composition according to claim 1, further comprising polyethylene glycol.

30. The method of claim 19, wherein the cross-linking agent is Eudragit.

31. The method of claim 22, wherein the polyaniline is emeraldine based polyaniline.

32. The method of claim 17, wherein the step of mixing includes the addition of polyethylene glycol.
